(19) United States
(12) Patent Application Publication
(10) Pub. No.: US 2006/0115442 A1
(43) Pub. Date: Jun. 1, 2006

(54) Facial cleansing composition

(75) Inventors: Melissa Iva Katz, Weston, CT (US); Megan Kathleen Hurley, Middletown, CT (US); Andre Marie Puleo, Stratford, CT (US); Paul Vinski, Danbury, CT (US)

Correspondence Address: UNILEVER INTELLECTUAL PROPERTY GROUP, 700 SYLVAN AVENUE,, BLDG C2 SOUTH, ENGLEWOOD CLIFFS, NJ, 07632-3100, US

(73) Assignee: Unilever Home & Personal Care USA, Division of Conopco, Inc.

(21) Appl. No.: 11/079,026

(22) Filed: Mar. 14, 2005

Related U.S. Application Data
(60) Provisional application No. 60/631,546, filed on Nov. 29, 2004.

(51) Int. Cl. A61K 8/81; A61K 8/72; A61K 8/73

(52) U.S. Cl. 424/70.11; 424/70.13; 424/70.27

(57) ABSTRACT

A facial cleansing composition is provided which includes the following ingredients: polyolefinic abrasive particles; a cationic surfactant; water; a hydrophobically modified cellulosic thickener; a water-soluble conditioning agent; a water-insoluble conditioning agent; and wherein the composition has a viscosity ranging from about 20,000 to about 200,000 cps.

### BACKGROUND OF THE INVENTION

1. Field of the Invention

The invention concerns a rinsable cosmetic composition for exfoliating skin.

2. The Related Art

Considerable foreign material normally reaches and attaches to the skin. This foreign material may include both exogenous and autochthonous soils. The exogenous soils include those which reach the skin unintentionally, or that which is intentionally applied, such as ointments or cosmetics. Autochthonous soils are the products of excretion of the sebaceous, eccrine and apocrine glands. Cells and flakes of the cornified epithelium are also being shed continuously. Both society and health demand that these soils be removed from time to time. For abnormal skin there must also be removal of pus, blood cells, serous exudates and crusts.

Bathing with a soap bar is the usual manner for removing foreign material from the skin. Soap is, however, harsh. Neither does soap provide the softening, lubricating, protective or even exfoliating properties that are necessary for full treatment of the skin.

U.S. Pat. No. 6,664,217 B1 (Puwada et al.) describes a toilet bar having simultaneous exfoliating and moisturizing properties. Although the exfoliant, surfactant and moisturizers are broadly described, the exemplified toilet bars rely on anionic syndets, particularly sodium cocoyl isethionate for cleansing activity. Polyethylene particles are utilized wherein at least 25% have particle size dimensions ranging from 100 to 600 micron.

U.S. Pat. No. 6,759,376 B2 (Zhang et al.) discloses oil-containing personal wash liquid compositions having particles of high refractive index and defined thickness, geometry and size. These rinse-off liquids are reported to enhance the shine of skin. All the exemplified surfactants are anionic, nonionic and amphoteric types.

Although the aforementioned patent documents report advances in exfoliating personal care compositions, none provide the benefit of gentle mechanical abrasion, extended foam, moisturization and also quick rinsability.

### SUMMARY OF THE INVENTION

A facial cleansing composition is provided which includes:
(i) from about 5 to about 30% of polyolefinic particles insoluble in water by weight of the composition;
(ii) from about 0.1 to about 20% of a cationic surfactant by weight of the composition;
(iii) from about 5 to about 90% of water by weight of the composition;
(iv) from about 0.01 to about 3% of a hydrophobically modified cellulosic thickener by weight of the composition;
(v) from about 0.01 to about 30% of a water soluble skin conditioning agent by weight of the composition;
(vi) from about 0.01 to about 30% of a water-insoluble skin conditioning agent by weight of the composition; and
wherein the composition has a viscosity ranging from about 20,000 to about 200,000 cps as measured on a Brookfield RVT Viscometer, spindle TC at 5 rpm for 1 minute at 23° C.

### DETAILED DESCRIPTION OF THE INVENTION

Now there has been found a composition which can exfoliate dry, dead skin cells in a cleansing cream based upon a cationic emulsion containing both hydrophilic and hydrophobic skin conditioners. These compositions utilize polyolefinic particles as the abrasive substance.

Accordingly, a first component of compositions according to the present invention is that of polyolefinic water-insoluble abrasive particles. Advantageously the abrasive particles have an average particle size ranging from about 50 to about 600 micron, preferably from about 100 to about 400 micron. The particle size range is the average of all particles with a measurement taken along a maximum length of each individual particle. Particularly suitable are low density polyethylene powders. One of these which has been useful in the present invention is Microscrub 80 PC beads sold by Micro Powders, Inc. Amounts of the abrasive particles may range from about 5 to about 30%, preferably from about 10 to about 20% by weight of the composition.

Efficient rinse-off from human skin of the compositions is achieved through a cationic surfactant. These are generally mono (C₈-C₂₂ alkyl or fatty amido) ammonium salts. The salt for purposes of this invention is an ammonium cation paired with an anion. Representative of the latter are anions selected from the group consisting of chloride, bromide, iodide, acetate, phosphate, nitrate, sulfate, methyl sulfate, ethyl sulfate, tosylate, lactate, citrate, glycolate and mixtures thereof.

Suitable cationic surfactants may be selected from the group consisting of cetyl ammonium chloride, cetyl ammonium bromide, lauryl ammonium chloride, lauryl ammonium bromide, stearyl ammonium chloride, stearyl ammonium bromide, cetyl dimethyl ammonium chloride, cetyl dimethyl ammonium bromide, lauryl dimethyl ammonium chloride, lauryl dimethyl ammonium bromide, stearyl dimethyl ammonium chloride, stearyl dimethyl ammonium bromide, cetyl trimethyl ammonium chloride, cetyl trimethyl ammonium bromide, lauryl trimethyl ammonium chloride, lauryl trimethyl ammonium bromide, stearyl trimethyl ammonium chloride, stearyl trimethyl ammonium bromide, lauryl dimethyl ammonium chloride, stearyl dimethyl ammonium chloride, tallow dimethyl ammonium chloride, stearamidopropyl dimethyl ammonium chloride, stearamidopropyl trimethylammonium methosulfate, stearamidopropyl PG-dimethyl ammonium methosulfate, behenamidopropyl PG-dimethylammonium chloride, and mixtures thereof. Most preferred is cetyl trimethylammonium chloride, known also as cetrimonium chloride available as Arquad 16-29®.

Amounts of the cationic surfactant may range from about 0.1 to about 20%, preferably from about 0.3 to about 10%, optimally from about 0.7 to about 1% by weight of the composition.

Compositions of this invention are emulsions, particularly with a water continuous phase. Accordingly, there will be present water in amounts which may range from about 5 to about 90%, preferably from about 50 to about 85%, optimally from 60 to 80% by weight of the composition.

Although anionic and nonionic surfactants are not excluded, the cationic surfactants generally will be the major emulsifier of the composition. In the event any anionic surfactant(s) are present, they will be in a weight ratio of total cationic to anionic surfactant(s) of at least about 2:1, preferably at least about 5:1, more preferably at least about 10:1, and optimally at least about 100:1.

Another component of compositions according to the present invention will be that of a hydrophobically modified cellulosic thickener. Particularly preferred cellulosics are the hydrophobically modified hydroxyethylcellulose. Especially useful for purposes of this invention is Natrosol Plus® available from the Hercules Corporation. The INCI name for the preferred material is cetyl hydroxyethylcellulose.

Amounts of the hydrophobically modified cellulosic thickener may range from about 0.01 to about 3%, preferably from about 0.1 to about 2%, optimally from about 0.3 to about 0.8% by weight of the composition.

Another component of compositions of the present invention is that of a water-soluble skin conditioning agent. Illustrative of these materials are glycerin, sorbitol, polyethylene glycol, triethylene glycol, polypropylene glycol, butylene glycol, hexylene glycol, isoprene glycol, dipropylene glycol, ethoxylated alcohols, urea and mixtures thereof.

Amounts of the water-soluble skin conditioning agent may range from about 0.01 to about 30%, preferably from about 0.5 to about 20%, optimally from about 1 to about 15% by weight of the composition.

A still further component of compositions according to the present invention are water-insoluble skin conditioning agents. These are generically represented by hydrocarbons, silicones and esters. Typical hydrocarbons are petrolatum, alpha-polyolefins, isoparaffins, polyisobutylene and combinations thereof. Useful silicones include cyclomethicone, dimethicone, amodimethicone, crosslinked emulsifying polysiloxane elastomers, crosslinked non-emulsifying polysiloxane elastomers (e.g. DC 9045), silicone waxes and combinations thereof. Suitable esters include synthetic esters and natural esters, the latter represented by triglycerides such as sunflower seed oil, borage oil, coriander seed oil and combinations thereof. Synthetic esters may include stearyl stearate, myristyl myristate, isopropyl myristate, capric/caprylic triglycerides and combinations thereof.

Amounts of the water-insoluble skin conditioning agent may range from about 0.01 to about 30%, preferably from about 0.5 to about 20%, optimally from about 1 to about 15% by weight of the composition.

Fatty acids and fatty alcohols having from 16 to 22 carbon atoms in the fatty group may be employed in compositions of this invention. Representative fatty acids include stearic acid, isostearic acid, linoleic acid, lauric acid, behenic acid, palmitic acid and combinations thereof. Representative fatty alcohols include stearyl alcohol, cetearyl alcohol, lauryl alcohol, behenyl alcohol and combinations thereof. Amounts of the fatty acid or alcohol may range from about 0.5 to about 20%, preferably from about 1 to about 10% by weight of the composition.

Cationic deposition aids may also be included in compositions of the present invention. These may be monomeric or polymeric ammonium salts. The monomeric are best illustrated by tricetylmonnium chloride, available commercially as ARQUAD 316®. Polymeric ammonium based deposition aids are materials such polyquaternium 10, polyquaternium 24 and Jaguar C13S®. Amounts of these materials may range from about 0.05 to about 3%, preferably from about 0.1 to about 1%, optimally from about 0.3 to about 0.8% by weight of the composition.

Advantageously, compositions of the present invention operate best with a total cationic material to that of total nonionic surfactant ranging from about 9:1 to about 1:2, preferably from about 9:1 to about 1:1, optimally from about 7:3 to about 1:1. The expression total cationic material refers to the sum of any cationic surfactant and cationic deposition aid.

Minor adjunct materials may also be incorporated. Especially useful are preservatives. Representative of these materials are methyl paraben, propyl paraben, imidazolidinyl urea, tetrasodium EDTA, tetrasodium edetate, iodopropynyl butylcarbamate, DMDM hydantoin, methylchloroisothiazolinone, methylisothiazolinone and combinations thereof. Amounts of the preservative may range from about 0.00001 to about 1%, preferably from about 0.001 to about 0.4%, optimally from about 0.001 to about 0.2% by weight of the composition and will be dependent upon the activity of the preservative.

Fragrances may be included at levels ranging from about 0.2 to about 2%, optimally from about 0.4 to about 1.5% by weight of the composition.

Other adjunct materials may include colorants, opacifiers and thickeners.

Herbal extracts, vitamins (vitamin A, vitamin B3, vitamin C, vitamin E and their derivatives), niacinamide, panthenol, retinol, pentapeptides, epidihydroandrosterone (DHEA), ceramides and combinations thereof. Amounts of these materials may range from about 0.00001 to about 3% by weight of the composition.

Compositions of the present invention will have a viscosity ranging from about 20,000 to about 200,000 cps, preferably from about 40,000 to 100,000, optimally from about 60,000 to about 80,000 cps. These viscosities are measured on a Brookfield RVT Viscometer, Spindle TC at 5 rpm measured for 1 minute at 23° C.

The term “comprising” is meant not to be limiting to any subsequently stated elements but rather to encompass non-specified elements of major or minor functional importance. In other words the listed steps, elements or options need not be exhaustive. Whenever the words “including” or “having” are used, these terms are meant to be equivalent to “comprising” as defined above.

Except in the operating and comparative examples, or where otherwise explicitly indicated, all numbers in this description indicating amounts of material ought to be understood as modified by the word “about”.

All documents referred to herein, including all patents, patent applications, and printed publications, are hereby incorporated by reference in their entirety in this disclosure.

The following examples will more fully illustrate the embodiments of this invention. All parts, percentages and proportions referred to herein and in the appended claims are by weight unless otherwise illustrated.

### EXAMPLES 1-8

Representative facial cleansing pastes according to the present invention are outlined below.

| | EXAMPLES (WEIGHT %) | | | | | | | |
|---|---|---|---|---|---|---|---|---|
| INGREDIENT | 1 | 2 | 3 | 4 | 5 | 6 | 7 | 8 |
| Microscrub 80 PC ® | 14.3 | 11.7 | 10.2 | 23.1 | 23.1 | 10.1 | 15.6 | 18.0 |
| Glycerin | 6.0 | 5.0 | 5.0 | 5.0 | 5.0 | 5.0 | 5.0 | 6.0 |
| Cetearyl Alcohol | 4.0 | 3.0 | 3.0 | 3.0 | 3.0 | 5.0 | 2.0 | 3.0 |
| Arquad 16-29 ® (Cetrimonium | 3.5 | 2.3 | 3.0 | 3.0 | 3.0 | 6.0 | 6.0 | 6.0 |
| Chloride, 29%) | | | | | | | | |
| Petrolatum | 0.5 | 1.0 | 2.0 | 2.0 | 2.0 | 2.0 | 2.0 | 2.0 |
| Stearyl Stearate | 0.5 | 0.5 | 1.5 | 1.5 | 1.0 | 1.0 | 1.0 | 2.0 |
| Arquad 316 ® (Tricetylmonnium | 0.4 | 0.5 | 0.4 | 0.4 | 0.8 | 0.8 | 0.8 | 0.8 |
| Chloride) | | | | | | | | |
| Silicone Fluid 350 (Dimethicone) | 0.2 | 0.2 | 0.3 | 0.3 | 0.3 | 0.2 | 0.2 | 0.2 |
| Glydant Plus ® | 0.2 | 0.2 | 0.2 | 0.2 | 0.2 | 0.2 | 0.2 | 0.2 |
| Fragrance | 0.2 | 0.2 | 0.2 | 0.2 | 0.2 | 0.2 | 0.2 | 0.2 |
| Natrosol Plus ® | 0.2 | 0.3 | 0.3 | 0.5 | 0.5 | 0.1 | 0.1 | 0.1 |
| Disodium EDTA | 0.05 | 0.05 | 0.05 | 0.05 | 0.05 | 0.05 | 0.05 | 0.05 |
| Water | Balance | Balance | Balance | Balance | Balance | Balance | Balance | Balance |

### EXAMPLE 9

An in vitro deposition experiment was conducted to determine the effect of varying ratios of cationic to nonionic surfactant. Six samples were evaluated, all of which except one was formulated with abrasive particles.

The evaluation protocol was as follows. Six pieces of porcine skin were utilized, one for each sample. All were washed in a 10% dishwashing solution, then dried in a dessicator until no further weight loss was observed. Each piece was weighed to the nearest 0.01 mg, and treated with one of the samples. Treatment consisted of: hydrating the skin for 5 minutes; blotting dry the skin; applying 5 μl of sample; washing for 15 seconds; rinsing for 30 seconds; and blotting dry the treated sample.

The pieces of porcine skin were then placed in a dessicator. Each piece was weighed after 3, 5, 7, 34 and 35 days. After 34 days there was no further weight loss.

Table I outlines the basic formula while Table II outlines variations to the basic formula.

TABLE I

| INGREDIENT | WEIGHT % |
|---|---|
| Microscrub 80 PC ® (Polyethylene) | 11.7 |
| Glycerin | 5.0 |
| Glycol Stearate and Stearamide AMP | 0-1.909* |
| Glyceryl Stearate | 0-0.891* |
| Cetearyl Alcohol | 3.0 |
| Arquad 16-29 ® (Cetrimonium Chloride, 29%) | 0-2.3* |
| Petrolatum | 1.0 |
| Stearyl Stearate | 0.5 |
| Arquad 316 ® (Tricetylmonium Chloride) | 0-0.5* |
| Silicone Fluid 350 (Dimethicone) | 0.2 |
| Glydant Plus ® | 0.2 |
| Fragrance | 0.2 |
| Natrosol Plus ® | 0.2 |
| Disodium EDTA | 0.05 |
| Extract | 0.011 |
| Water | balance |

*Varied per Table II.

TABLE II

| | | | % Nonionic Surfactant | | |
|---|---|---|---|---|---|
| | % Cationic Material | | Glycol Stearate | | Ratio |
| Sample | Arquad | Arquad | and Stearamide | Glyceryl | Cationic to |
| No. | 16-29 ® | 316 ® | AMP | Stearate | Nonionic |
| 1 | 2.3 | 0.5 | 0 | 0 | 100:0 |
| 2 | 2.3 | 0.5 | 1.909 | 0.891 | 50:50 |
| | | | | | (no PE |
| | | | | | abrasive) |
| 3 | 2.3 | 0.5 | 0.1909 | 0.0891 | 90:10 |
| 4 | 2.3 | 0.5 | 0.01909 | 0.00891 | 99:1 |
| 5 | 2.3 | 0.5 | 0.818 | 0.382 | 70:30 |
| 6 | 0 | 0 | 1.194 | 0.557 | 0:100 |

Results of the porcine skin test are reported in Table III.

TABLE III

Treatment Time (Days)

| Sample No. | 3 | 5 | 7 | 34 |
|---|---|---|---|---|
| 1 | 0.287 | 0.260 | 0.251 | 0.219 |
| 2 | 0.491 | 0.418 | 0.402 | 0.072 |
| 3 | 0.632 | 0.541 | 0.478 | 0.078 |
| 4 | 0.598 | 0.545 | 0.502 | 0.358 |
| 5 | 0.592 | 0.485 | 0.401 | 0.008 |
| 6 | 0.704 | 0.599 | 0.554 | 0.482 |

Changes in weight gain reported in Table III revealed the highest for the sample which does not contain any cationic material (Sample 6). Lowest weight gains occurred where ratios of cationic to nonionic were 70:30, 50:50 and 90:10, respectively. These data reveal the advantage of having combinations of both cationic and nonionic components, with the majority being cationic material.

What is claimed is:

1. A facial cleansing comprising:
(i) from about 5 to about 30% of polyolefinic particles insoluble in water by weight of the composition;
(ii) from about 0.1 to about 20% of a cationic surfactant by weight of the composition;
(iii) from about 5 to about 90% of water by weight of the composition;
(iv) from about 0.01 to about 3% of a hydrophobically modified cellulosic thickener by weight of the composition;
(v) from about 0.01 to about 30% of a water soluble skin conditioning agent by weight of the composition;
(vi) from about 0.01 to about 30% of a water-insoluble skin conditioning agent by weight of the composition; and
wherein the composition has a viscosity ranging from about 20,000 to about 200,000 cps as measured on a Brookfield RVT Viscometer, spindle TC at 5 rpm for 1 minute at 23° C.

2. The composition according to claim 1 wherein the particles are polyethylene.

3. The composition according to claim 2 wherein the cationic surfactant is cetrimmonium salt.

4. The composition according to claim 1 wherein the hydrophobically modified cellulosic thickener is cetyl hydroxyethylcellulose.

5. The composition according to claim 1 wherein the water soluble conditioning agent is glycerin.

6. The composition according to claim 1 wherein the water-insoluble skin conditioning agent is selected from the group consisting of hydrocarbons, silicones, esters and combinations thereof.

7. The composition according to claim 1 wherein no anionic surfactant is present.

8. The composition according to claim 1 wherein an anionic surfactant is present, total cationic to anionic surfactants being in a weight ratio of at least about 2:1.

9. The composition according to claim 1 further comprising from about 0.05 to about 3% of a cationic deposition aid.

10. The composition according to claim 9 wherein the cationic surfactant and if present any cationic deposition aid together are in a weight ratio relative to all nonionic surfactant present in an amount from about 9:1 to about 1:1.
